(19) United States
(12) Patent Application Publication
(10) Pub. No.: US 2009/0186942 A1
(43) Pub. Date: Jul. 23, 2009

(54) NOVEL NUTRACEUTICAL AND PHARMACEUTICAL COMPOSITIONS AND USE THEREOF FOR THE TREATMENT, CO-TREATMENT OR PREVENTION OF INFLAMMATORY DISORDERS

(76) Inventors: Daniel Raederstorff, Flaxlanden (FR); Joseph Schwager, Basel (CH); Goede Schueler, Eimeldingen (DE)

Correspondence Address: NIXON & VANDERHYE, PC, 901 NORTH GLEBE ROAD, 11TH FLOOR, ARLINGTON, VA, 22203, US

(21) Appl. No.: 12/279,516

(22) PCT Filed: Feb. 13, 2007
(86) PCT No.: PCT/EP07/01227

§ 371 Date: Aug. 14, 2008

(30) Foreign Application Priority Data
Feb. 16, 2006 (EP) 06003156.4

(51) Int. Cl. A61K 31/192; C07C 65/03; A61P 29/00

(52) U.S. Cl. 514/568; 562/475

(57) ABSTRACT

The invention relates to novel compositions comprising a phenolic compound as well as to the use of these compositions as a medicament, in particular for the manufacture of a nutraceutical or pharmaceutical composition for the treatment, co-treatment or prevention of inflammatory disorders, such as arthritis, asthma, inflammatory bowel diseases, inflammatory diseases of the skin (e.g., psoriasis, eczema, atopic dermatitis, sunburn) and chronic inflammatory disorders, such as atherosclerosis, heart diseases, metabolic syndrome X, cancer, Alzheimer's disease and pre-stages thereof such as mild cognitive impairment. Also, the invention relates to the use of a cosmetic composition comprising a phenolic compound for the cosmetic treatment, co-treatment or prevention of inflammation of the skin, in particular for the cosmetic treatment, co-treatment or prevention of sunburn or of impure skin.

The present invention relates to novel compositions comprising phenolic compounds as well as to the use of these compositions as a medicament, in particular as a medicament for the treatment, co-treatment or prevention of inflammatory disorders.

Inflammatory disorders are one of the most important health problems in the world. Inflammation is in general a localized protective response of the body tissues to invasion of the host by foreign material or injurious stimuli. The causes of inflammation can be infectious agents such as bacteria, viruses, and parasites; or physical agents such as burns or radiation; or chemicals like toxins, drugs or industrial agents; or immunological reactions such as allergies and autoimmune responses or conditions associated with oxidative stress.

Inflammation is characterized by pain, redness, swelling, heat, and eventual loss of function of the affected area. These symptoms are the results of a complex series of interactions taking place between the cells of the immune system. The response of the cells results in an interacting network of several groups of inflammatory mediators: Proteins (e.g. cytokines, enzymes (e.g. proteases, peroxydase), major basic protein, adhesion molecules (ICAM, VCAM), lipid mediators (e.g. eicosanoids, prostaglandins, leukotrienes, platelet activating factor (PAF)), reactive oxygen species (e.g. hydroperoxides, superoxyde anion O₂⁻, nitric oxide (NO) etc). However, many of those mediators of inflammation are also regulators of normal cellular activity. Thus, deficiencies of inflammatory reactions lead to a compromised host (i.e. infection) while uncontrolled and thus chronic inflammation leads to inflammatory diseases mediated in part by the excessive production of several of the above mentioned mediators.

Acute and chronic inflammation resulting from an excessive biosynthesis of inflammatory mediators is involved in numerous inflammatory disorders such as arthritis (e.g. osteoarthritis, rheumatoid arthritis), asthma, inflammatory bowel diseases, inflammatory diseases of the skin (e.g. psoriasis, eczema, atopic dermatitis, sunburn) and chronic inflammatory disorders, such as atherosclerosis, heart diseases, metabolic syndrome X, cancer, Alzheimer's disease and pre-stages thereof such as mild cognitive impairment.

Rheumatoid arthritis is a chronic inflammatory disease of the joints. For example, arthritis includes rheumatoid arthritis, spondylorathropathies, gouty arthritis, osteoarthritis, systemic lupus erythematosus and juvenile arthritis. Asthma and rheumatoid arthritis are characterised at the molecular level by chronically unbalanced expression of cytokines, chemokines, kinins and their receptors, adhesion molecules, and inflammatory enzymes. Psoriasis is one of the most common skin problems, affecting 1-3% of the human population. Inflammatory bowel disease is a general term used to describe gastrointestinal tract diseases such as ulcerative colitis and Crohn's disease.

Beside the process of intravascular lipid deposition, inflammatory reactions of the endothelial (i.e. blood vessel) wall are considered to critically contribute to atherosclerosis i.e. atheroma formation. Atherosclerosis results from vascular injury which triggers inflammation. Activated macrophages, T-lymphocytes, and mast cells are present in atherosclerotic plaques. Monocyte/macrophage and lymphocyte activation leads to the release of eicosanoids and cytokines which are implicated in endothelial damage, as well as in the formation and eventually the rupture of atherosclerotic plaques. Finally, circulating inflammatory markers such as C-reactive protein (CRP), fibrinogen, and interleukins are increased in groups at high-risk of coronary artery diseases (CAD). Several clinical trials indicate that elevated CRP concentration correlates with increased risk of coronary, and vascular, events. Thus inflammation appears to play an important role in the initiation and progression of atheroma formation.

Inflammatory disorders are also associated with the pathophysiology of Alzheimer's disease. There is evidence of inflammation in the brain of patients with Alzheimer's disease, as it is characterized by increased levels of cytokines and activated microglial cells. Thus, inflammation is not only involved in the classical inflammatory disorders (e.g. arthritis, asthma, bowel diseases) but is also associated with many chronic inflammatory disorders (e.g. atherosclerosis, heart diseases, metabolic syndrome X, cancer, Alzheimer disease).

Inflammatory events are also associated with the pathophysiology of different types of cancer (e.g. gastric and intestinal cancers, melanomas). Increased levels of prostaglandins have been found in cancers of breast, colon, lung and pancreas in humans.

Two mains classes of drugs, the corticosteroid and the nonsteroidal anti-inflammatory drugs (NSAIDs) are used to treat inflammatory disorders. NSAIDs and corticosteroids provide essentially symptomatic relief. Use of corticosteroids has declined due to a growing concern about the serious side effects of prolonged use.

NSAIDs are among the most widely used drugs, primarily for the treatment of pain and inflammatory disorders, in particular for the treatment of arthritis.

Epidemiological studies have suggested that patients taking NSAIDs have a lower risk of developing Alzheimer's disease than those not taking NSAIDs. A protective effect of NSAIDs suggests that the cyclooxygenases might be involved in the neurodegenerative process.

Epidemiological studies showed a significant reduction in the risk of colorectal, gastric, esophageal, and breast cancers among people who take non-steroidal anti-inflammatory drugs (NSAIDs) compared with those not taking NSAIDs. In animal models, NSAIDs significantly reduced tumor development.

However, chronic use of NSAIDs when treating chronic diseases such as arthritis, is limited by severe side-effects like serious gastrointestinal complications, renal toxicity or asthmatic reactions.

Therefore, there is a need for new anti-inflammatory agents with weak or no side effects. Patients with inflammatory diseases have a special interest in treatment considered as “natural” with mild anti-inflammatory effects and without major side effects, which can be used for disease prevention and as adjuvant treatment.

It is the object of the present invention to address such need.

This object is achieved by the invention by a composition comprising a phenolic compound of formula (1)

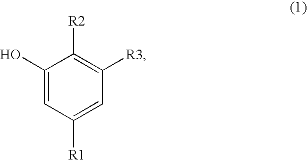

wherein R¹ stands for H, OH or for methoxy, wherein R² stands for H or COOH and wherein R³ stands for a saturated, a mono- or a polyunsaturated C₁₄, C₁₅, C₁₆ or C₁₇ alkylchain. If the alkylchain is monounsaturated, the double bond can be of (E) or (Z) configuration. If the alkylchain is polyunsaturated, the double bonds can be independently of each other either of (E) or (Z) configuration. Preferably in all embodiments of the invention R³ stands for a saturated, a mono- or a polyunsaturated alkylchain being either of structures (a)-(k):

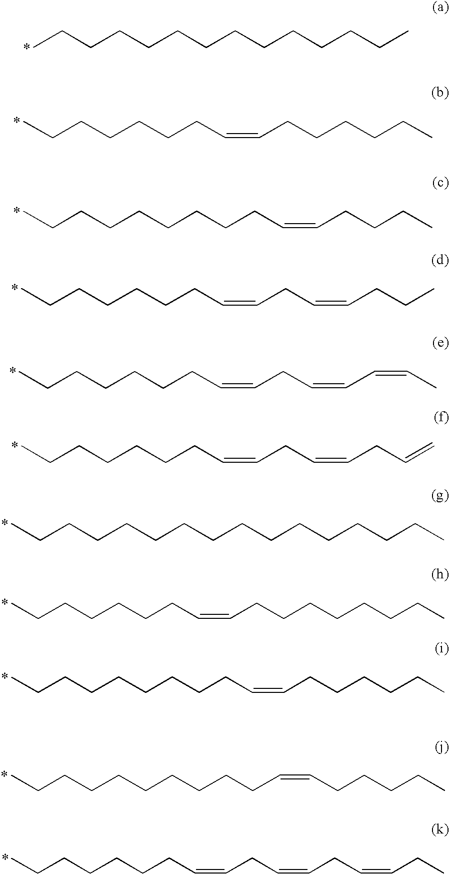

Within the framework of the invention, with * is indicated the attachment point of the R³ group to formula (1).

It has surprisingly been found that the phenolic compound of formula (1) is an anti-inflammatory agent. Therefore, the composition of the present invention may be especially useful in the treatment, co-treatment and prevention of inflammatory disorders, such as heart disease, multiple sclerosis, osteo- and rheumatoid arthritis, atherosclerosis, and osteoporosis.

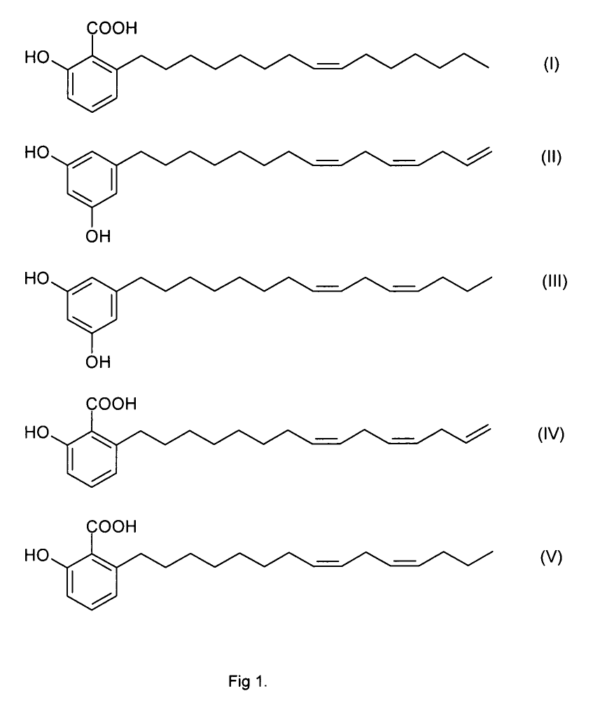

The composition of the present invention is especially suitable for the treatment, co-treatment and prevention of arthritis, in particular osteoarthritis and rheumatoid arthritis. Therefore, the composition of the present invention may have one or more of the following properties: it reduces joint inflammation, it maintains and/or increases joint health, it prevents joint stiffness, it increases mobility, it provides supple and/or flexible joints, it lubricates the joints, it relieves arthritis pain, it relieves pain associated with joint inflammation, it decreases joint swelling, it lessens joint problems, it provides joint care. More preferably the phenolic compound of formula (1) is selected from the group of Ginkgoic acid (I), Cardoltriene (II), Cardoldiene (III), (15:3)-Anacardic acid (IV), and (15:2)-Anacardic acid (V). Compounds (I), (II), (III), (IV), (V) can be found in FIG. 1. Even more preferably, the phenolic compound of formula (1) is selected from the group of Cardoltriene (II) and Cardoldiene (III). Most preferably, the phenolic compound is Cardoldiene (III).

The phenolic compound of formula (1) is preferably used in a concentration so that the daily consumption by a human adult (weighing about 70 kg) is in the range of from 1 mg/day to 2000 mg/day, preferably from 5 mg/day to 500 mg/day. A food or beverage preferably comprises between 0.2 mg and 1000 mg of phenolic compound of formula (1) per serving. If the nutraceutical composition is a pharmaceutical formulation such formulation may preferably comprise a phenolic compound of formula (1) in an amount between 0.5 mg and 2000 mg per dosage unit, e.g., per capsule or tablet, or between 1 mg per daily dose and 3000 mg per daily dose of a liquid formulation.

Therefore, the invention also relates to a composition comprising between 0.5 mg and 3000 mg, preferably between 1 and 2000 mg, more preferably between 1 and 500 mg of phenolic compound of formula (1).

The composition of the invention expressly also encompasses an extract comprising a phenolic compound of formula (1) such as for instance an—preferably organic phase or supercritical fluid—extract of the cashew plant or a part of the cashew plant, for example of the cashew apple. Furthermore, the invention also encompasses compositions comprising phenolic compound of formula (1) according to the invention with the definitions and preferences as given above in the form of an extract, in particular in the form of an extract obtainable from the plant material of the cashew plant or a part of the cashew plant, for example of the cashew apple such as for instance a organic phase extract.

The phenolic compound of formula (1) may be synthesized or extracted and/or purified by methods known to the person skilled in the art.

The phenolic compounds of formula (1) are preferably derived from the cashew plant that may be obtained from conventional and commercially available sources such as growers.

A number of phenolic compounds of formula (1) are found in Anacardium occidentale, the cashew nut, the cashew nut shell, the cashew apple, and in various Toxicodendron species like T. radicans, T diversilobum. Furthermore, some of the phenolic compounds of formula (1) can be found in Rhus verniciflua, Melanorrhoea usitata, Amorpha fruticosa or in Cajanus cajan and can be isolated thereof by methods known to a skilled person in the art. Ginkgoic acid (I) may for example also be obtained from Ginkgo bilboa (like leaves and fruits).

The phenolic compounds employed herein may be prepared by a number of methods known in the art. The mentioned plants may be processed by any suitable means to obtain the compositions described. For example, cashew apple may be extracted to obtain a mixture. The phenolic compounds may be obtained directly from the mixture or the mixture may be fractionated and/or purified to obtain the phenolic compounds. The compositions may be fractionated and/or purified by a number of methods known to the person skilled in the art. Examples of fractionating methods include partitioning with an organic solvent, chromatography, for example high pressure liquid chromatography (HPLC) or the use of supercritical fluids.

All compounds mentioned in FIG. 1. can for example be obtained by extraction of dried plant material of Anacardium occidentale with methanol:MTB (9:1) and by subsequent fractionation of the thus obtained crude extract by preparative HPLC in a buffered solvent system.

In a different aspect, the invention also relates to the phenolic compound of formula (1) and/or composition of the invention, for use as a medicament. More in particular the invention relates to use of this compound and/or composition for the manufacture of a nutraceutical or pharmaceutical composition for the treatment, co-treatment or prevention of inflammatory disorders, more preferably of arthritis, most preferably of rheumatoid arthritis or osteoarthritis. Also, the invention relates to a method for treatment, co-treatment and prevention of inflammatory disorders, preferably of arthritis, most preferably of osteoarthritis, in animals including humans said method comprising the step of administering an effective amount of phenolic compound of formula (1) and/or the composition according to the invention to animals including humans, which are in need thereof.

In the framework of the invention, with animals is meant all animals, including mammals, examples of which include humans. Preferred examples of mammals beside humans include dogs, dromedaries, camels, elephants, and horses.

In another aspect, the invention relates to use of a phenolic compound of formula (1) and/or a composition according to the invention, for the manufacture of a nutraceutical or pharmaceutical composition.

In yet another aspect, the invention relates to a composition according to the invention, wherein the composition is a food or beverage or a supplement composition for a food or beverage.

In another aspect, the invention relates to a composition according to the invention, wherein the composition is a pharmaceutical composition further comprising a pharmaceutically acceptable carrier.

The nutraceutical and pharmaceutical compositions according to the present invention may be in any galenic form that is suitable for administering to the animal body including the human body, more in particular in any form that is conventional for oral administration, e.g. in solid form, for example as (additives/supplements for) food or feed, food or feed premixes, fortified food or feed, tablets, pills, granules, drageés, capsules, and effervescent formulations such as powders and tablets, or in liquid form, for instance in the form of solutions, emulsions or suspensions, for example as beverages, pastes and oily suspensions. The pastes may be filled into hard or soft shell capsules. Examples for other application forms are forms for transdermal, parenteral, topical or injectable administration. The nutraceutical and pharmaceutical compositions may be in the form of controlled (delayed) release formulations. Examples of pharmaceutical compositions also include compositions suitable for topical application and transdermal absorption of the phenolic compound, such as crèmes, gels, sprays, dry sticks, powders etc. Moreover, a multi-vitamin and mineral supplement may be added to the nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

A person skilled in the art knows which carriers can be used as pharmaceutically acceptable carriers. Examples of such pharmaceutically acceptable carriers are both inorganic and organic carrier materials, suitable for oral/parenteral/injectable administration and include water, gelatin, gum arabic, lactose, starch, magnesium stearate, talc, vegetable oils, and the like.

Besides a phenolic compound of formula (1) and a pharmaceutically acceptable carrier, the pharmaceutical composition according to the present invention, may further contain conventional pharmaceutical additives and adjuvants, excipients or diluents, including, but not limited to, water, gelatin of any origin, vegetable gums, ligninsulfonate, talc, sugars, starch, gum Arabic, vegetable oils, polyalkylene glycols, flavoring agents, preservatives, stabilizers, emulsifying agents, buffers, lubricants, colorants, wetting agents, fillers, and the like.

Examples of fortified food are cereal bars, chewing gum and bakery items, such as cakes and cookies.

Beverages encompass non-alcoholic and alcoholic drinks as well as liquid preparation to be added to drinking water and liquid food. Non-alcoholic drinks are for instance soft drinks, sport drinks, fruit juices, such as for example orange juice, apple juice and grapefruit juice; lemonades, teas, near-water drinks and milk based drinks, such as for example yoghurt drinks. Examples of liquid food include soups and dairy products, for instance yoghurt.

The phenolic compound of formula (1) may be used in combination with other nutraceutical compositions or therapeutic agents know to those skilled in the art for treatment or prevention of inflammatory disorder by administration prior to, simultaneously with or following the administration of phenolic compound of formula (1). In another embodiment of the invention, the phenolic compound of formula (1) may be incorporated into cosmetic or dermatological compositions (the latter compositions are a specific type of pharmaceutical compositions) such as skin care preparations for the treatment, co-treatment or prevention of inflammation of the skin, in particular of sunburn caused by UV-radiation or such as for example skin care preparations for the treatment, co-treatment or prevention of impure skin. Examples of impure skin include pimples, acne and other skin impurities with an inflammatory aspect.

Therefore, the invention also relates to the use of the cosmetic composition for the cosmetic treatment, co-treatment or prevention of inflammation of the skin, in particular for the cosmetic treatment, co-treatment or prevention of sunburn. The invention also relates to the use of the phenolic compound of formula (1) and/or a composition according to the invention for the manufacture of a dermatological composition for the treatment, co-treatment or prevention of inflammation of the skin, in particular of sunburn or of impure skin. Also, the invention relates to a method for the treatment, co-treatment or prevention of inflammation of the skin, in particular of sunburn in humans or of impure skin such as for example acne, said method comprising the step of administering an effective amount of the dermatological composition according to the invention to humans, which are in need thereof. Also, the invention relates to a method for cosmetic treatment co-treatment or prevention of inflammation of the skin, in particular of sunburn or of impure skin by a cosmetic composition according to the invention. Sunburn prevention is preferably achieved with topical application of the phenolic compound of formula (1) or a composition according to the invention, preferably in combination with suitable light screening agents.

The cosmetic or dermatological compositions according to the invention may be in the form of a suspension or dispersion in solvents or fatty substances, or alternatively in the form of an emulsion or micro emulsion (in particular of O/W or W/O type, O/W/O or W/O/W-type, wherein O stands for organic phase and wherein W stands for water phase), such as a cream, a paste, a lotion, a thickened lotion or a milk, a vesicular dispersion in the form of an ointment, a gel, a solid tube stick or an aerosol mousse, and may be provided in the form of a mousse, foam or a spray foams, sprays, sticks or aerosols or wipes. Examples of cosmetic or dermatological compositions are skin care preparations, in particular, body oils, body lotions, body gels, treatment creams, skin protection ointments, moisturizing gels, moisturizing sprays, revitalizing body sprays and after sun preparations or sunscreen formulations.

The cosmetic or dermatological composition for the treatment, co-treatment or prevention of inflammation of the skin, such as for example sunburn or impure skin may be in a form that is conventional for oral administration, examples of which are described above, and also include beauty foods and supplements.

The cosmetic or dermatological compositions of the invention, for instance sunscreen formulations or after sun preparations, may further comprise the usual cosmetic respectively dermatological adjuvants and/or additives, such as for example preservatives/antioxidants, fatty substances/oils, water, organic solvents, silicones, thickeners, softeners, emulsifiers, additional light screening agents, antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, light stabilizers, insect repellants, skin tanning agents, skin whitening agents, antibacterial agents, preservatives, or any other ingredients usually formulated into cosmetics.

Light screening agents which may be incorporated into cosmetic or dermatological compositions of the invention for instance sunscreen formulations are advantageously selected from UV-A, UV-B, UV-C and/or broadband filters. Examples of UV-B or broad spectrum screening agents, i.e. substances having absorption maximums between about 290 and 340 nm may be organic or inorganic compounds. Organic UV-B or broadband screening agents are e.g. acrylates such as 2-ethylhexyl 2-cyano-3,3-diphenylacrylate (octocrylene, PARSOL® 340), ethyl 2-cyano-3,3-diphenylacrylate and the like; camphor derivatives such as 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor, therephthalidene dicamphor sulfonic acid and the like; Cinnamate derivatives such as ethylhexyl methoxycinnamate (PARSOL® MCX), ethoxyethyl methoxycinnamate, diethanolamine methoxycinnamate (PARSOL® Hydro), isoamyl methoxycinnamate and the like as well as cinnamic acid derivatives bond to siloxanes; p-aminobenzoic acid derivatives, such as p-aminobenzoic acid, 2-ethylhexyl p-dimethylaminobenzoate, N-oxypropylenated ethyl p-aminobenzoate, glyceryl p-aminobenzoate; benzophenones such as benzophenone-3, benzophenone-4,2,2′,4,4′-tetrahydroxy-benzophenone, 2,2′-dihydroxy-4,4′-dimethoxybenzophenone and the like; esters of benzalmalonic acid such as di-(2-ethylhexyl) 4-methoxybenzalmalonate; esters of 2-(4-ethoxy-anilinomethylene)propandioic acid such as 2-(4-ethoxy anilinomethylene) propandioic acid diethyl ester as described in the European Patent Publication EP 0895 776; organosiloxane compounds containing benzmalonate groups as described in the European Patent Publications EP 0358584 B1, EP 0538431 B1 and EP 0709080 A1 such as polysilicone-15 (PARSOL® SLX); drometrizole trisiloxane (Mexoryl XL); imidazole derivatives such as e.g. 2-phenyl benzimidazole sulfonic acid and its salts (PARSOL®HS). Salts of 2-phenyl benzimidazole sulfonic acid are e.g. alkali salts such as sodium- or potassium salts, ammonium salts, morpholine salts, salts of primary, sec. and tert. amines like monoethanol amine salts, diethanol amine salts and the like; salicylate derivatives such as isopropylbenzyl salicylate, benzyl salicylate, butyl salicylate, ethylhexyl salicylate (PARSOL® EHS, NEO Heliopan OS), isooctyl salicylate or homomethyl salicylate (homosalate, PARSOL® HMS, NEO Heliopan OS) and the like; triazine derivatives such as ethylhexyl triazone (Uvinul T-150), diethylhexyl butamido triazone (Uvasorb HEB). Encapsulated UV-filters such as encapsulated ethylhexyl methoxycinnamate (Eusolex UV-pearls) or microcapsules loaded with UV-filters as e.g. disclosed in EP 1471995 and the like. Inorganic compounds are pigments such as microparticulated TiO₂, ZnO and the like. The term “microparticulated” refers to a particle size from about 5 nm to about 200 nm, particularly from about 15 nm to about 100 nm. The TiO₂ particles may also be coated by metal oxides such as e.g. aluminum or zirconium oxides or by organic coatings such as e.g. polyols, methicone, aluminum stearate, alkyl silane. Such coatings are well known in the art.

Examples of broad spectrum or UV A screening agents i.e. substances having absorption maximums between about 320 and 400 nm may be organic or inorganic compounds e.g. dibenzoylmethane derivatives such as 4-tert. butyl-4′-methoxydibenzoyl-methane (PARSOL® 1789), dimethoxydibenzoylmethane, isopropyldibenzoylmethane and the like; benzotriazole derivatives such as 2,2′-methylene-bis-(6-(2H-benzotriazole-2-yl)-4-(1,1,3,3,-tetramethylbutyl)-phenol (TINOSORB M) and the like; bis-ethylhexyloxyphenol methoxyphenyl triazine (Tinosorb S) and the like; phenylene-1,4-bis-benzimidazolsulfonic acids or salts such as 2,2-(1,4-phenylene)bis-(1H-benzimidazol-4,6-disulfonic acid) (Neoheliopan AP); amino substituted hydroxybenzophenones such as 2-(4-Diethylamino-2-hydroxy-benzoyl)-benzoic acid hexylester (Uvinul A plus) as described in the European Patent Publication EP 1046391; Ionic UV-A filters as described in the International Patent Publication WO2005080341 A1. Pigments such as microparticulated ZnO or TiO2 and the like. The term “microparticulated” refers to a particle size from about 5 nm to about 200 nm, particularly from about 15 nm to about 100 nm. The particles may also be coated by other metal oxides such as e.g. aluminum or zirconium oxides or by organic coatings such as e.g. polyols, methicone, aluminum stearate, alkyl silane. Such coatings are well known in the art.

As dibenzoylmethane derivatives have limited photostability it may be desirable to photostabilize these UV-A screening agents. Thus, the term “conventional UV-A screening agent” also refers to dibenzoylmethane derivatives such as e.g. PARSOLS® 1789 stabilized by, e.g. 3,3-Diphenylacrylate derivatives as described in the European Patent Publications EP 0 514 491 B1 and EP 0 780 119 A1; Benzylidene camphor derivatives as described in the U.S. Pat. No. 5,605,680; Organosiloxanes containing benzmalonate groups as described in the European Patent Publications EP 0358584 B1, EP 0538431 B1 and EP 0709080 A1.

Active ingredients which may be included in the cosmetic or dermatological compositions of the invention are for example vitamins and derivatives thereof, for example tocopherol, tocopherolacetate, ascorbic acid, ascorbyl phosphate, vitamin Q, D, and K, retinol, retinal, retinoic acid, retinol acetate, retinol palmitate, biotin, carotinoid derivatives such as beta carotene, lycopene, asthaxanthin, vegetable extracts, antibacterial ingredients, instable amino acids comprising dipeptides, oligopeptides and polypeptides such as methionine, cysteine, cystine, tryptophan, phenylalanine, tyrosine, phenols, polyphenols or flavanoids, bisabolol, allantoin, phytantriol, panthenol, AHA acids, ubiquinones such as Coenzyme Q 10, ceramides, pseudoceramides, essential oils, plant extracts deoxyribonucleic acid.

The necessary amounts of the cosmetic and dermatological adjuvants, additives and/or additional active ingredients can, based on the desired product, easily be chosen by a person skilled in the art and will be illustrated in the examples, without being limited hereto.

In yet another aspect, the invention also relates to a composition of the invention, wherein the composition is a cosmetic or dermatological composition comprising a cosmetic respectively dermatological adjuvant and/or a cosmetic respectively dermatological additive and/or a cosmetic respectively dermatological additional active ingredient.

The cosmetic or dermatological compositions comprise the phenolic compound of formula (1) in an effective amount. The term “effective amount” is preferably at least 0.01% by weight of the composition. Preferably, the compositions comprise the phenolic compound of formula (1) in an amount between 0.01 wt.-% and 20 wt.-%, more preferably between 0.05 and 10 wt.-%, still more preferably between 0.1 and 5 wt.-%.

The invention will now be elucidated by way of the following examples, without however being limited thereto.

### EXAMPLE 1

### Soft Gelatin Capsules

Soft gelatin capsules are prepared by conventional procedures providing a dose of a phenolic compound of formula (1) of 50 mg. A suitable daily dose is 1 to 5 capsules. Other ingredients: glycerol. Water, gelatine, vegetable oil

### EXAMPLE 2

### Hard Gelatin Capsules

Hard gelatin capsules are prepared by conventional procedures providing a dose of phenolic compound of formula (1) of 20 mg. A suitable daily dose is 1 to 5 capsules.

Other ingredients:

Fillers: lactose or cellulose or cellulose derivatives q.s.
Lubricant: magnesium stearate if necessary (0.5%)

### EXAMPLE 3

### Tablet

Tablets are prepared by conventional procedures providing as active ingredient 20 mg of a phenolic compound of formula (1) per tablet, and as excipients microcrystalline cellulose, silicone dioxide (SiO₂), magnesium stearate, crospovidone NF (which is a disintegration agent) ad 500 mg.

### EXAMPLE 4

### Soft Drink

A soft drink containing COMPOUND may be prepares as follows:

A soft drink is prepared from the following ingredients:

| | |
|---|---|
| ingredient | [g] |

A. juice concentrates and water soluble flavours

| | |
|---|---|
| 60.3° Brix, 5.15% acidity | 657.99 |
| 43.5° Brix, 32.7% acidity | 95.96 |
| Orange flavour, water soluble | 3.43 |
| Apricot flavour, water soluble | 6.71 |
| water | 26.46 |

B. color

| | |
|---|---|
| a-carotene 10% CWS | 0.89 |
| water | Ad 100 |

C. Acid and antioxidant

| | |
|---|---|
| Ascorbic acid | 4.11 |
| Citric acid anhydrous | 0.69 |
| water | 43.18 |

D. stabilizers

| | |
|---|---|
| pectin | 0.20 |
| Sodium benzoate | 2.74 |
| water | 65.60 |

E. oil soluble flavours

| | |
|---|---|
| Orange flavour, oil soluble | 0.34 |
| Orange oil distilled | 0.34 |

F. active ingredient

| | |
|---|---|
| cardoldiene | Amount providing 500 mg |

Fruit juice concentrates and water-soluble flavours are mixed without incorporation of air. The color is dissolved in deionized water. Ascorbic acid and citric acid are dissolved in water. Sodium benzoate is dissolved in water. The pectin is added under stirring and dissolved while boiling. The solution is cooled down. Orange oil and oil soluble flavours are premixed. The active ingredient as mentioned under F is stirred into the fruit juice concentrate mixture of A.

In order to prepare the soft drinks all components A-F are mixed together before homogenizing using a Turrax and then a high-pressure homogenizer (p₁=200 bar, p₂=50 bar).

### EXAMPLE 5

### Preparation of a Dermatological Composition Comprising a Phenolic Compound of Formula (1) (Treatment Cream) which May be Used for (Cosmetic) Treatment of Inflammation of the Skin Caused by Sunburn

A treatment cream may be prepared with the following ingredients, in the following amounts:

| | Ingredients | INCI Nomenclature | % w/w |
|---|---|---|---|
| A | Glyceryl Myristate | Glyceryl Myristate | 2.00 |
| | Phenolic compound of | | 0.05-25 |
| | formula (1) | | |
| | Cetyl Alcohol | Cetyl Alcohol | 0.50 |
| | Myritol 318 | Caprylic/Capric Triglyceride | 5.00 |
| | Crodamol DA | Diisopropyl Adipate | 5.00 |
| | Vitamin E acetate | Tocopheryl Acetate | 2.00 |
| | Butylated | BHT | 0.05 |
| | Hydroxytoluene | | |
| | Phenonip | Phenoxyethanol & Methylparaben | 0.60 |
| | | & Ethylparaben & Propylparaben | |
| | | & Butylparaben | |
| | Edeta BD | Disodium EDTA | 0.10 |
| | AMPHISOL K | Potassium Cetyl Phosphate | 2.00 |
| B | Water deionized | Aqua | ad 100 |
| | 1,2-Propylene Glycol | Propylene Glycol | 2.00 |
| | D-PANTHENOL 75 L | Panthenol | 2.00 |
| | Ethanol | Ethanol | 5.00 |
| | Allantoin | Allantoin | 0.20 |
| | Carbopol ETD 2001 | Carbomer | 0.30 |
| C | KOH 10% sol. | Potassium Hydroxide | 1.50 |
| D | Perfume | Perfume | q.s. |

Procedure: Heat part A) and B) to 85° C. while stirring. When homogeneous, add part B) to A) under agitation. Cool to about 45° C. while stirring. Add part C). Homogenize at 11000 rpm to achieve a small particle size. Cool to ambient temperature while stirring. Then add part D).

### EXAMPLE 6

### Inhibition of the Production of Inflammatory Mediators

The anti-inflammatory effects of structurally related substances of A. occidentale (cashew apple) like cardoldiene, cardoltriene, anacardiac acid and ginkoic acid were determined in cellular assays. The substances were isolated from an extract of Anacardium occidentale. The extract was obtained by extraction of dried plant material of Anacardium occidentale with methanol:methyl tert butyl ether (MTB) (9:1) and by subsequent fractionation of the thus obtained extracted by preparative HPLC in a buffered solvent system. These extracts were tested in cellular inflammatory systems and the IC₅₀ of the mixture of substances was determined (see below).

Pure substances were then further assayed in vitro in cellular cell systems. The inhibition of the synthesis of nitric oxide and/or pro-inflammatory prostaglandins (PG) by the compounds was measured. PGE₂ plays a critical role in the inflammation process, while nitric oxide (NO) is a hallmark of inflammation in various chronic inflammatory diseases including various forms of arthritis, gastro-intestinal diseases and metabolic syndrome X. The effects of compounds on the inflammatory response were tested in cellular assays using a murine macrophage indicator cell line, RAW267.4. The cells were purchased from ATCC (Manassas, Va., USA) and cultured in DMEM containing streptomycin/penicillin, non-essential amino acids and 10% fetal calf serum (FCS). In order to test a large range of concentration of compounds, cells (˜50,000/well) were seeded into flat-bottomed microtiter plates and cultured for one day. Cells were then starved in complete medium containing 0.25% FCS (D-025). After overnight culture, medium was removed and replaced by 100 μL of D-025 containing the test compounds at twice the final concentration. Subsequently, 100 μL of D-025 containing 2 μg/ml LPS was added (i.e. final LPS concentration of 1 μg/ml) and the cells cultured for 24 hours.

Substances were tested in a concentration range from 0.2 to 50 μM in two-fold dilution steps. Extracts were tested from 0.2 to 50 mg/L. All treatments were done in duplicates and several experimental series were done for each treatment. Concentrations of nitrite which was rapidly formed from nitric oxide released by cells were determined by the Griess reaction using sodium nitrite as standard. Briefly, 50 μl of supernatant was mixed with Griess reagent 1 (25 μL) and Griess reagent 2 (25 μL), centrifuged and the optical density at 540 nm determined. PGE₂ secreted into the cell culture medium was determined by EIA obtained from Cayman Chemicals (Ann Harbor, Wis., USA) and used according to the manufacturer's instructions. All determinations were done in duplicates and at various dilutions of the culture supernatant. IC₅₀ values were calculated using a two-parametric least-square fitting equation [y=A+((B−A)/(1+((C−x)̂D))] for best-fit curves (Excel fit software program).

The effects of compounds on inhibition of two inflammatory parameters are given in the Tables below. The extract of A. occidentale potently inhibited both nitric oxide and PGE₂ production; the observed IC₅₀ values reflect an elevated concentration of bio-active compounds. Three of the five substances identified in A. occidentale extracts (i.e cardoldiene, cardoltriene, [15:3] anarcardic acid) potently reduced the production of nitric oxide (NO) with an IC₅₀ of <˜20 μmol/L. The impact on PGE₂ production was even more important in the case of cardoldiene and cardoltriene. The tested substances exerted a biological activity that was better or similar to that of resveratrol or EGCG, which have widely demonstrated anti-inflammatory effects.

TABLE
IC₅₀ values for A. occidentale extracts

| Extrakt | IC₅₀ Nitric Oxide | IC₅₀ PGE₂ |
|---|---|---|
| A. occidentale | 2.2 mg/L | 0.7 mg/L |

TABLE
IC₅₀ values for single substances

| Compound | IC₅₀ Nitric Oxide | | IC₅₀ PGE₂ | |
|---|---|---|---|---|
| Cardoldiene | 8.9 ± 1.5 | μmol/L | 2.5 ± 1.4 | μmol/L |
| Cardoltriene | 4.9 ± 0.8 | μmol/L | 0.9 ± 0.2 | μmol/L |
| (15:3)-Anacardic acid | 7.4 ± 0.4 | μmol/L | 26.6 ± 21.1 | μmol/L |
| (15:2)-Anacardic acid | 82.4 ± 6.7 | μmol/L | 43.1 ± 5.5 | μmol/L |
| Ginkoic acid | >100 | μmol/L | 57.8 | μmol/L |
| Resveratrol | 31 ± 2 | μmol/L | 24 ± 2 | μmol/L |
| EGCG | 33 ± 2 | μmol/L | 35 +/− 3 | μmol/L |

### EXAMPLE 7

### Modulation of the Expression Levels of Inflammatory Genes

The effect of the compounds has been evaluated on the level of the expression of genes that are involved in the inflammatory response. These comprise e.g. the following genes TNF-α, IL-6, MIP1β and NF-κB1 and NF-κBp49, the latter two of which are transcription factors involved in the regulation of inflammatory gene expression. RAW 264.7 cells were stimulated in the presence of different concentrations of substances (indicated in the FIGURE). After 4 hours, RNA was extracted and the expression of genes determined by quantitative RT-PCR as described (Richard, N., Porath, D., Radspieler, A. and Schwager, J. Effects of resveratrol, piceatannol, tri-acetoxystilbene, and genistein on the inflammatory response of human peripheral blood leukocytes. Mol Nutr Food Res 2005. 49: 431-442). As an example, data are shown for (15:3)-anarcardic acid, that significantly reduced TNF-α and MIP-1β. Collectively, the results demonstrate effects of the compounds on the production of inflammatory mediators and on distinct genes involved in the inflammatory response.

TABLE
Effect of (15:3)-anarcardic acid on the expression of inflammatory
genes (TNF-α, IL-6, macrophage inflammatory protein 1 [MIP-1],
transcription factors involved in the regulation of inflammatory gene
expression [NF-κB1, NF-κBp49]). The level of mRNA
for a given gene is expressed relative to the level observed in cells
that were stimulated with LPS only. Values lower than 100%
indicate that the substance had an inhibitory effect on the
expression of the relevant gene.

| | % expression in the | % expression in the presence of |
|---|---|---|
| | presence of 12.5 μM/L | 6.25 μM/L |
| Gene | (15:3) anarcardic acid | (15:3) anarcardic acid |
| TN\tilde{F}-α, | 54 | 76 |
| IL-6, | 71 | 65 |
| MIP\tilde{1}-α | 41 | 58 |
| NF-κB1 | 78 | 86 |
| N\tilde{F}-κBp49 | 57 | 74 |

### EXAMPLE 8

### Effect of Cardoldiene on Carrageenan-Induced Paw Edema in Rats

The anti-inflammatory activity of cardoldiene was evaluated in vivo in the carrageenan-induced paw edema model. This model has long been used to assess the anti-inflammatory properties of agents that inhibit prostaglandins, such as nonsteroidal anti-inflammatory drugs (NSAIDs). The model causes time-dependent edema formation following carrageenan administration into the subplantar surface of a rat paw.

Twenty male Wistar (Han) rats weighing 120 to 150 g were randomized in two groups. They were housed in a temperature (21±3° C.) and relative humidity (30-80%) controlled room with a 12-h light/dark cycle. They had ad libitum access to filtered tap-water and standard pelleted laboratory chow throughout the study and were housed 4 to 5 per cage and at least a 5 day acclimatization period was observed before any testing.

Cardoldiene (200 mg/kg) dissolved in corn oil (in a volume of 5 mL/kg) or vehicle alone were administered by the oral route in a coded and random order after an overnight fast. Thirty minutes later, inflammation is induced by subplantar injection of 0.05 ml of a 1.5% carrageenan suspension into the right hindpaw. The left hindpaw was injected with 0.05 ml physiological saline. The paw volume of each rat was measured in mL at two time points once 1.5 h and once 3.5 h after the injection of carrageenan. The right paw edema volume is determined by the difference between the right hindpaw volume (inflamed paw) and the left (non-inflamed) hindpaw volume. The anti-inflammatory effect on edema volume in treated-groups was expressed as percent (%) inhibition [(mean of vehicle-treated group paw edema volume−mean of the treated group paw edema volume)/mean of vehicle-treated group paw edema volume)×100]. For results see table below.

TABLE
Pharmacological effects of cardoldiene after oral administration
on carrageenan-induced paw edema in rats

| | Paw edema volume (ml) | | |
|---|---|---|---|
| | vehicle-treated | Cardoldiene treated | % Inhibition by |
| Time (hours) | animals | animals | cardoldiene |
| 1.5 | 0.69 | 0.63 | 9 |
| 3 | 0.85 | 0.72 | 15 |

All data of paw edema volume are expressed in mL as Mean of 10 rats in each group. % inhibition vs vehicle-treated group is calculated.

It is shown that cardoldiene (200 mg/kg) inhibited the mean paw edema volume 1.5 h and 3.5 h after the carrageenan injection as compared to the control group treated with the vehicle. Therefore, cardoldiene has anti-inflammatory action in mammals.

### EXAMPLE 9

### O/W Sun Milk

| | Ingredients | INCI Nomenclature | % w/w |
|---|---|---|---|
| A) | PARSOL SLX | Dimethico | 6.00 |
| | | DiethylbenzalmalonatePolysilicone-15 | |
| | Neo Heliopan AP | | 3.00 |
| | Tinosorb S | Hydrogenated Cocoglycerides | 3.00 |
| | Lanette O | Cetearyl Alcohol | 2.00 |
| | Myritol 318 | Caprylic/capric Triglyceride | 6.00 |
| | Mineral oil | Mineral oil | 2.00 |
| | Vitamin E acetate | Tocopheryl Acetate | 1.00 |
| | Prisorine 3515 | Isostearyl Alcohol | 4.00 |
| B) | Edeta BD | Disodium EDTA | 0.10 |
| | Phenonip | Phenoxyethanol & Methylparaben & | 0.60 |
| | | Ethylparaben & Propylparaben & | |
| | | Butylparaben | |
| | Amphisol K | Potassium Cetyl Phosphate | 2.00 |
| | Water deionized | Aqua | ad100 |
| | 1,2-Propylene | Propylene Glycol | 5.00 |
| | Glycol | | |
| | Carbopol 981 | Carbomer | 0.30 |
| | Tinosorb M | Methylene Bis-Benzotriazolyl | 6.00 |
| | | Tetramethylbutylphenol | |
| | KOH 10% | Potassium Hydroxyde | 2.10 |
| | solution | | |
| C) | Phenolic | For example the structures | 0.05-25 |
| | compound | as given in FIG. 1 | |

### Procedure:

Heat part A) and B) to 85° C. while stirring. When homogeneous, add part B) to A) under agitation. Cool to ambient temperature while stirring and add part C). Homogenize to achieve a small particle size.

### EXAMPLE 10

### Sun Milk Waterproofed

| | Ingredients | INCI Nomenclature | % w/w |
|---|---|---|---|
| A) | PARSOL SLX | Polysilicone-15 | 6.00 |
| | PARSOL 1789 | Butyl Methoxydibenzoylmethane | 2.00 |
| | PARSOL 5000 | 4-Methylbenzylidene Camphor | 4.00 |
| | Uvinul T 150 | Ethylhexyltriazone | 2.00 |
| | Silicone | Dimethicone | 1.00 |
| | DC 200/350 cs | | |
| | Lanette O | Cetearyl Alcohol | 2.00 |
| | Softisan 100 | Hydrogenated Coco-Glycerides | 3.00 |
| | Tegosoft TN | C12-15 Alkyl Benzoate | 6.00 |
| | Cetiol B | Dibutyl Adipate | 7.00 |
| | Vitamin E acetate | Tocopheryl Acetate | 2.00 |
| | BHT | BHT | 0.05 |
| | Edeta BD | Disodium EDTA | 0.10 |
| | Phenonip | Phenoxyethanol & Methylparaben & | 0.60 |
| | | Ethylparaben & Propylparaben & | |
| | | Butylparaben | |
| | Amphisol | Cetyl Phosphate DEA | 2.00 |
| B) | Water deionized | Aqua | ad 100 |
| | Propylene Glycol | Propylene Glycol | 5.00 |
| | Carbopol 980 | Carbomer | 0.30 |
| | KOH (10% sol.) | Potassium Hydroxide | 1.50 |
| C) | Phenolic compound | For example the | 0.05-25 |
| | | structures as given in FIG. 1 | |

### Procedure:

Heat part A) and B) to 85° C. while stirring. When homogeneous, add part B) to A) under agitation. Cool to ambient temperature while stirring and add part C). Homogenize to achieve a small particle size.

### EXAMPLE 11

### Sun Milk for Babies and Children

| | Ingredients | INCI Nomenclature | % w/w |
|---|---|---|---|
| A) | Tegosoft TN | C12-15 Alkyl Benzoate | 5.00 |
| | Silicone 2503 | Stearyl Dimethicone | 2.00 |
| | Cosmetic Wax | | |
| | Cetyl Alcohol | Cetyl Alcohol | 1.00 |
| | Butylated | BHT | 0.05 |
| | Hydroxytoluene | | |
| | Estol GMM 3650 | Glyceryl Myristate | 4.00 |
| | Edeta BD | Disodium EDTA | 0.10 |
| | Phenonip | Phenoxyethanol & Methylparaben & | 0.60 |
| | | Ethylparaben & Propylparaben & | |
| | | Butylparaben | |
| | Amphisol A | Cetyl Phosphate | 2.00 |
| B) | Water deionized | Aqua | ad 100 |
| | Carbopol 980 | Carbomer | 0.6 |
| | Glycerine | Glycerine | 3.00 |
| | KOH sol. 10% | Potassium Hydroxide | 2.4 |
| C) | Phenolic compound | For example the structures | 0.05-25 |
| | | as given in FIG. 1 | |

### Procedure:

Heat part A) and B) to 85° C. while stirring. When homogeneous, add part B) to A) under agitation. Cool to ambient temperature while stirring and add part C). Homogenize to achieve a small particle size.

### EXAMPLE 12

### High Protective Sun Milk

| | Ingredients | INCI Nomenclature | % w/w |
|---|---|---|---|
| A) | PARSOL SLX | Polysilicone-15Dimethico | 6.00 |
| | | Diethylbenzalmalonate | |
| | PARSOL 1789 | Butyl Methoxydibenzoylmethane | 2.00 |
| | PARSOL 5000 | 4-Methylbenzylidene Camphor | 4.00 |
| | Uvinul T 150 | | 2.00 |
| | Silicone | Dimethicone | 1.00 |
| | DC 200/350 cs | | |
| | Lanette O | Cetearyl Alcohol | 2.00 |
| | Softisan 100 | Hydrogenated Coco-Glycerides | 3.00 |
| | Tegosoft TN | C12-15 Alkyl Benzoate | 6.00 |
| | Cetiol B | Dibutyl Adipate | 7.00 |
| | Vitamin E acetate | Tocopheryl Acetate | 2.00 |
| | BHT | BHT | 0.05 |
| | Edeta BD | Disodium EDTA | 0.10 |
| | Phenonip | Phenoxyethanol & Methylparaben & | 0.60 |
| | | Ethylparaben & Propylparaben & | |
| | | Butylparaben | |
| | Amphisol K | Potassium Cetyl Phosphate | 2.00 |
| B) | Water deionized | Aqua | ad 100 |
| | Propylene Glycol | Propylene Glycol | 5.00 |
| | Carbopol 980 | Carbomer | 0.30 |
| | KOH (10% sol.) | Potassium Hydroxide | 1.50 |
| C) | phenolic compound | For example the structures | 0.05-25 |
| | | as given in FIG. 1 | |
| D) | Perfume | Perfume | q.s. |

Procedure: Heat part A) and B) to 85° C. while stirring. When homogeneous, add part B) to A) under agitation. Cool to ambient temperature while stirring and add part C) and D). Homogenize to achieve a small particle size.

### EXAMPLE 13

### Water-Free Sun Gel

| | Ingredients | INCI Nomenclature | % w/w |
|---|---|---|---|
| A) | PARSOL MCX | Ethylhexyl Methoxycinnamate | 6.00 |
| | PARSOL 1789 | Butyl Methoxydibenzoylmethane | 4.00 |
| | PARSOL 5000 | 4-Methylbenzylidene Camphor | 4.00 |
| | Uvasorb HEB | Diethylhexyl Butamido Triazone | 1.50 |
| | Uvinul A plus | | 2.00 |
| | Vitamin E acetate | Tocopheryl Acetate | 1.50 |
| | Tegosoft TN | C12-15 Alkyl Benzoate | 9.00 |
| | Elefac I-205 | Ethylhexyldodecyl Neopentanoate | 2.00 |
| | Alcohol | Alcohol | ad 100 |
| | Isopropyl Alcohol | Isopropyl Alcohol | 20.00 |
| B) | Klucel MF | Hydroxypropylcellulose | 2.00 |
| C) | phenolic compound | For example the structures as given in | 0.05-25 |
| | | FIG. 1 | |
| D) | perfume | | q.s. |

### Procedure:

Mix part A) and B) while stirring. When homogeneous, add part C) and D) under agitation.

### EXAMPLE 14

### Sun Gel

| | Ingredients | INCI Nomenclature | % w/w |
|---|---|---|---|
| A) | Pemulen TR-2 | Acrylates/C10-30 Alky Acrylate | 0.60 |
| | | Crosspolymer | |
| | Phenonip | Phenoxyethanol & Methylparaben & | 0.60 |
| | | Ethylparaben & Propylparaben & | |
| | | Butylparaben | |
| | Edeta BD | Disodium EDTA | 0.1 |
| | Aqua | Aqua | ad 100 |
| B) | PARSOL 1789 | Butyl Methoxydibenzoylmethane | 4.00 |
| | PARSOL 340 | Octocrylene | 3.00 |
| | Tegosoft TN | C12-15 Alkyl Benzoate | 15.00 |
| | Antaron V-216 | PVP/Hexadecene Copolymer | 1.00 |
| | Vitamin E acetate | Tocopheryl Acetate | 0.50 |
| | Butylated | BHT | 0.05 |
| | Hydroxytoluene | | |
| | Cremophor RH 410 | PEG-40 Hydrogenated Castor Oil | 0.50 |
| | Tris Amino | Tromethamine | 0.50 |
| C) | phenolic compound | For example the structures | 0.05-25 |
| | | as given in FIG. 1 | |
| D) | Perfume | Perfume | q.s. |

### Procedure:

Heat part A) and B) to 85° C. while stirring. When homogeneous, add part B) to A) under agitation. Cool to ambient temperature while stirring and add part C) and D). Homogenize to achieve a small particle size.

### EXAMPLE 15

### High Protection WO Sun Milk

| | Ingredients | INCI Nomenclature | % w/w |
|---|---|---|---|
| A) | PARSOL 1789 | Butyl Methoxydibenzoylmethane | 2.00 |
| | PARSOL 5000 | 4-Methylbenzylidene Camphor | 4.00 |
| | Uvinul T 150 | Ethylhexyl Triazone | 2.00 |
| | Uvinul TiO2 | Titanium Dioxide and | 5.00 |
| | | Trimethoxycaprylylsilane | |
| | Arlacel P 135 | PEG-30 Dipolyhydroxystearate | 2.00 |
| | Tegosoft TN | C12-15 Alkyl Benzoate | 5.00 |
| | Cosmacol EMI | Di-C12-13 Alkyl Malate | 6.00 |
| | Miglyol 840 | Propylene Glycol | 6.00 |
| | | Dicaprylate/Dicaprate | |
| | Butylated | BHT | 0.05 |
| | Hydroxytoluene | | |
| | Phenonip | Phenoxyethanol & Methylparaben | 0.60 |
| | | & Ethylparaben & Propylparaben | |
| | | & Butylparaben | |
| B) | Deionized water | Aqua | ad 100 |
| | Glycerin | Glycerin | 5.00 |
| | Edeta | Disodium EDTA | 0.1 |
| | NaCl | Sodium Chloride | 0.30 |
| C) | PARSOL HS | Phenylbenzyimidazole Sulphonic | 4.00 |
| | | Acid | |
| | Water | Aqua | 20.00 |
| | Triethanolamine 99%. | Triethanolamine | 2.50 |
| D) | phenolic compound | For example the structures | 0.05-25 |
| | | as given in FIG. 1 | |
| E) | Perfume | | q.s. |

Procedure: Heat part A), B) and C) to 85° C. while stirring. When homogeneous, add part B) and C) to A) under agitation. Cool to ambient temperature while stirring and add part D) and E). Homogenize to achieve a small particle size.

### EXAMPLE 16

### W/O Milk with Pigments

| | Ingredients | INCI Nomenclature | % w/w |
|---|---|---|---|
| A) | Cremophor WO 7 | PEG-7 Hydrogenated Castor Oil | 6.00 |
| | Elfacos ST 9 | PEG-45/Dodecyl Glycol Copolymer | 2.00 |
| | PARSOL 1789 | Butyl Methoxydibenzoylmethane | 3.00 |
| | Tinosorb S | | 5.00 |
| | PARSOL 5000 | 4-Methylbenzylidene Camphor | 4.00 |
| | microfine ZnO | Zinc Oxide | 2.00 |
| | Microcrystalline | Microcrystalline | 2.00 |
| | Wax | | |
| | Miglyol 812 | Caprylic/capric Triglyceride | 5.00 |
| | Vitamin E acetate | Tocopheryl Acetate | 1.00 |
| | Jojoba oil | Simmondsia Chinensis Seed Oil | 5.00 |
| | Edeta BD | Disodium EDTA | 0.10 |
| | Butylated | BHT | 0.05 |
| | Hydroxytoluene | | |
| | Phenonip | Phenoxyethanol & Methylparaben & | 0.60 |
| | | Ethylparaben & Propylparaben & | |
| | | Butylparaben | |
| B) | Water deionized | Aqua | ad 100 |
| | Glycerin | Glycerin | 5.00 |
| C) | Neo Heliopan AP | | 2.00 |
| | Water deionized | Aqua | 20.00 |
| | KOH 10% solution | Potassium Hydroxide | 4.00 |
| D) | phenolic compound | For example the structures | 0.05-25 |
| | | as given in FIG. 1 | |
| E) | Perfume | Perfume | q.s. |

Procedure: Heat part A), B) and C) to 85° C. while stirring. When homogeneous, add part B) and C) to A) under agitation. Cool to ambient temperature while stirring and add part D) and E). Homogenize to achieve a small particle size.

### EXAMPLE 17

### Protective Day Cream with Vitamin C

| | Ingredients | INCI Nomenclature | % w/w |
|---|---|---|---|
| A) | PARSOL SLX | Polysilicone-15Dimethico | 4.00 |
| | | Diethylbenzalmalonate | |
| | PARSOL 1789 | Butyl Methoxydibenzoylmethane | 1.50 |
| | Glyceryl Myristate | Glyceryl Myristate | 2.00 |
| | Cetyl Alcohol | Cetyl Alcohol | 0.50 |
| | Myritol 318 | Caprylic/Capric Triglyceride | 5.00 |
| | Crodamol DA | Diisopropyl Adipate | 5.00 |
| | Vitamin E acetate | Tocopheryl Acetate | 2.00 |
| | Butylated | BHT | 0.05 |
| | Hydroxytoluene | | |
| | Phenonip | Phenoxyethanol & Methylparaben | 0.60 |
| | | & Ethylparaben & Propylparaben | |
| | | & Butylparaben | |
| | Edeta BD | Disodium EDTA | 0.10 |
| | Amphisol K | Potassium Cetyl Phosphate | 2.00 |
| B) | Water deionized | Aqua | ad 100 |
| | 1,2-Propylene Glycol | Propylene Glycol | 2.00 |
| | D-Panthenol 75 L | Panthenol | 2.00 |
| | Ethanol | Ethanol | 5.00 |
| | Allantoin | Allantoin | 0.20 |
| | Carbopol ETD 2001 | Carbomer | 0.30 |
| | KOH 10% sol. | Potassium Hydroxide | 1.50 |
| C) | Water | Aqua | 10.00 |
| | Stay-C 50 | Sodium Ascorbyl Phosphate | 0.50 |
| D) | phenolic compound | For example the structures | 0.05-25 |
| | | as given in FIG. 1 | |
| E) | Perfume | Perfume | q.s. |

1. Composition comprising a phenolic compound of formula (1) 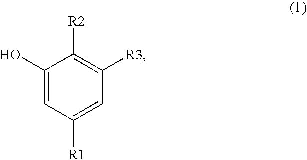
wherein R¹ stands for H, OH or methoxy, wherein R² stands for H or COOH and
wherein R³ stands for a saturated, a mono- or a polyunsaturated Ci₄, C₁₅, Ci₆ or Ci₇ alkylchain.

2. Composition according to claim 1, wherein R³ stands for a saturated, mono- or polyunsaturated alkylchain being either of structures (a)-(k)

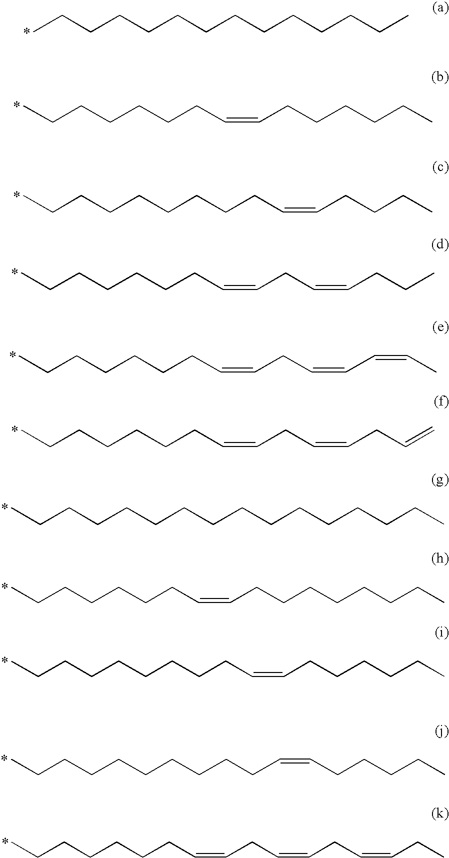

3. Composition according to claim 1, wherein the phenolic compound of formula (1) is selected from the group of Ginkgoic acid (I), Cardoltriene (II), Cardoldiene (III), (15:3)-Anacardic acid (IV), and (15:2)-Anacardic acid (V).

4. Composition according to claim 1, wherein the phenolic compound of formula (1) is selected from the group of Cardoltriene (II) and Cardoldiene (III).

5. Composition according to claim 1, wherein the dosage of the phenolic compound of formula (1) is between 0.2 mg to 3000 mg.

6. Composition according to claim 1, for use as a medicament.

7. Use of a phenolic compound of formula (1) 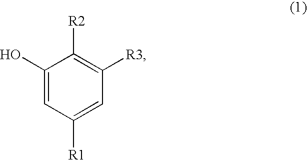
wherein R¹ stands for H, OH or methoxy, wherein R² stands for H or COOH and wherein R³ stands for a saturated, mono- or polyunsaturated C₎₄, Ci₅, Ci₆ or Ci₇ alkylchain and/or a composition according to claim 1, for the manufacture of a nutraceutical or pharmaceutical composition for the treatment, co-treatment or prevention of inflammatory disorders.

8. Use according to claim 7, wherein R³ stands for a saturated, mono- or polyunsaturated alkylchain being either of structures (a)-(k)

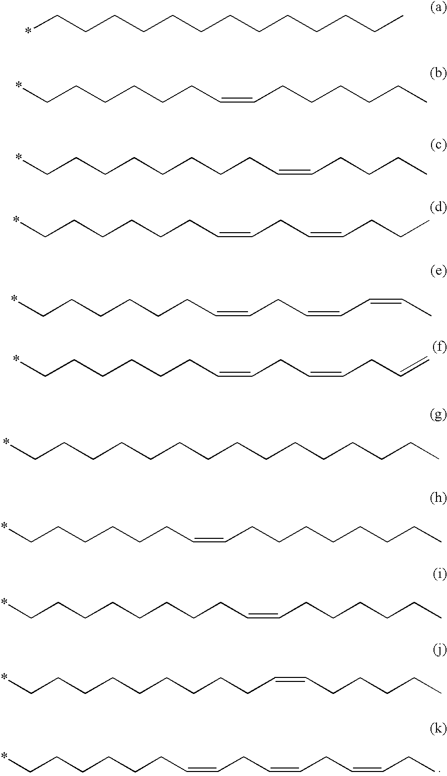

9. Use according to claim 7, wherein the inflammatory disorder is arthritis.

10. Use of a composition according to claim 1, for the manufacture of a nutraceutical or pharmaceutical composition.

11. Composition according to claim 1, wherein the composition is a food or a beverage or a supplement composition for a food or beverage.

12. Composition according to claim 1, wherein the composition is a pharmaceutical composition further comprising a pharmaceutically acceptable carrier.

13. Composition according to claim 1, wherein the composition is a cosmetic or dermatological composition further comprising a cosmetic respectively dermatological adjuvant and/or a cosmetic respectively dermatological additive and/or a cosmetic respectively dermatological additional active ingredient.

14. Use of the cosmetic composition of claim 13 for the cosmetic treatment, co-treatment or prevention of inflammation of the skin, in particular for the cosmetic treatment, co-treatment or prevention of sunburn or of impure skin.

15. Use of a phenolic compound of formula (1) 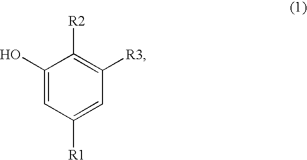
wherein R¹ stands for H, OH or methoxy, wherein R² stands for H or COOH and wherein R stands for a saturated, a mono- or a polyunsaturated Ci₄, Ci₅, Ci₆ or Ci₇ alkylchain or of a composition according to claim 1 for the manufacture of a dermatological composition for the treatment, co-treatment or prevention of inflammation of the skin, in particular for the treatment, co-treatment or prevention of sunburn or of impure skin.

16. Use according to claim 15, wherein R³ stands for a saturated, mono- or polyunsaturated alkylchain being either of structures (a)-(k)

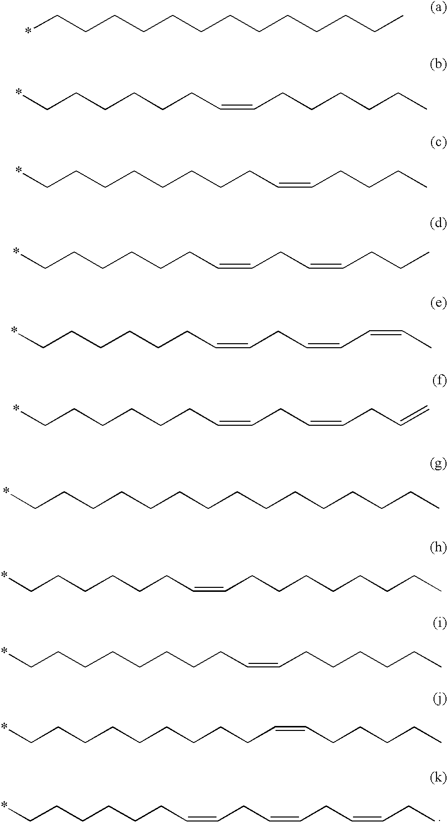

17. Method for treatment, co-treatment or prevention of inflammatory disorders in animals including humans said method comprising the step of administering an effective amount of a phenolic compound of formula (1) 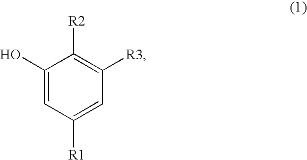
wherein R¹ stands for H, OH or methoxy, wherein R² stands for H or COOH and wherein R³ stands for a saturated, mono- or polyunsaturated Ci₄, Ci₅, Ci₆ or Ci₇ alkylchain or a composition according to claim 1 to animals including humans, which are in need thereof.

18. Method according to claim 17, wherein R³ stands for a saturated, mono- or polyunsaturated alkylchain being either of structures (a)-(k)

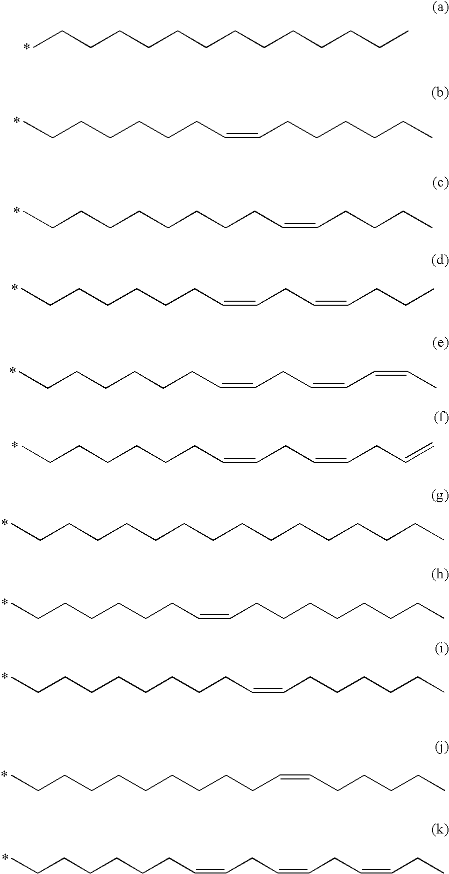

19. Method according to claim 17, wherein the inflammatory disorder is arthritis.

20. Method according to claim 17, wherein the inflammatory disorder is inflammation of the skin, in particular sunburn or acne.
